(19) United States
(12) Patent Application Publication
(10) Pub. No.: US 2004/0142362 A1
(43) Pub. Date: Jul. 22, 2004

(54) Inferring gene regulatory networks from time-ordered gene expression data using differential equations

(76) Inventors: Satoru Miyano, Tokyo (JP); Seiya Imoto, Tokyo (JP); Michiel De Hoon, Tokyo (JP)

Correspondence Address: D. Benjamin Borson, Fliesler Dubb Meyer & Lovejoy LLP, Ste. 400, 4 Embardcadero Center, San Francisco, CA, 94131, US

(21) Appl. No.: 10/722,033

(22) Filed: Nov. 25, 2003

Related U.S. Application Data
(60) Provisional application No. 60/428,827, filed on Nov. 25, 2002.

(51) Int. Cl. C12Q 1/68; G06F 19/00; G01N 33/48; G01N 33/50

(52) U.S. Cl. 435/6; 702/20

(57) ABSTRACT

Embodiments of methods are provided that can be used to estimate network relationships between genes of an organism using time course expression data and a set of linear differential equations. Aikaike's Information Criterion and mask tools can be used to reduce the number of elements in a matrix by determining which elements are zero or not significantly changed under the conditions of the study. Maximum likelihood estimation and new statistical methods are used to evaluate the significance of a proposed network relationship.

### RELATED APPLICATION

This application claims priority under 35 U.S.C. §119(e) to U.S. Provisional Application Serial No.: 60/428,827 filed Nov. 25, 2002. This application is herein incorporated fully by reference.

### FIELD OF THE INVENTION

This invention relates to methods for determining relationships between genes of an organism. In particular, this invention includes new methods for inferring gene regulatory networks from time course gene expression data using a linear system of differential equations.

### BACKGROUND

One of the most important aspects of current research and development in the life sciences, medicine, drug discovery and development and pharmaceutical industries is the need to develop methods and devices for interpreting large amounts of raw data and drawing conclusions based on such data. Bioinformatics has contributed substantially to the understanding of systems biology and promises to produce even greater understanding of the complex relationships between components of living systems. In particular, with the advent of new methods for rapidly detecting expressed genes and for quantifying expression of genes, bioinformatics can be used to predict potential therapeutic targets even without knowing with certainty, the exact roles a particular gene(s) may play in the biology of an organism.

Simulation of genetic systems is a central topic of systems biology. Because simulations can be based on biological knowledge, a network estimation method can support biological simulation by predicting or inferring previously unknown relationships.

In particular, development of microarray technology has permitted studies of expression of a large number of genes from a variety of organisms. A large amount of raw data can be obtained from a number of genes from an organism, and gene expression can be studied by intervention either by mutation, disease or drugs. Finding that a particular gene's expression is increased in a particular disease or in response to a particular intervention may lead one to believe that that gene is directly involved in the disease process or drug response. However, in biological organisms genes rarely are independently regulated by any such intervention, in that many genes can be affected by a particular intervention. Because a large number of different genes may be so affected, understanding the cause and effect relationships between genes in such studies is very difficult. Thus, much effort is being expended to develop methods for determining cause and effect relationships between genes, which genes are central to a biological phenomenon, and which genes' expression(s) are peripheral to the biological process under study. Although such peripheral gene's expression may be useful as a marker of a biological or pathophysiological condition, if such a gene is not central to physiological or pathophysiological conditions, developing drugs based on such genes may not be worth the effort. In contrast, for genes identified to be central to a process, development of drugs or other interventions may be crucial to developing treatments for conditions associated with altered expression of genes.

Microarray technology allows gene expression levels to be measured for a large number of genes at the same time. Microarray analysis can be carried out using complementary DNA (cDNA) easily, but RNA microarrays can also be used to study gene expression. While the amount of available gene expression data has been increasing rapidly, techniques to analyze such data is still in development. Increasingly, mathematical methods are being employed to determine relationships between expressed genes. However, accurately deriving a gene regulatory network from gene expression data can be difficult.

In time-ordered gene expression measurements, the temporal pattern of gene expression can be investigated by measuring the gene expression levels at a small number of points in time. Periodically varying gene expression levels have, for instance, been measured during the cell cycle of the yeast Saccharomyces cerevisiae (see Ref. 1). Gene responses to a slowly changing environment have been measured during a diauxic shift of the same yeast (see Ref. 2). Other experiments measured temporal gene expression patterns in response to an abrupt change in the environment of the organism. As an example, the gene expression response was measured of the cyanobacterium Synechocystis sp. PCC 6803 after to sudden shift in the intensity of external light (see Refs. 3 and 4).

Several methods have been proposed to infer gene interrelations from expression data. In cluster analysis (see Refs. 2, 5 and 6), genes are grouped together based on the similarity between their gene expression profiles. Inferring Boolean or Bayesian networks from measured gene expression data has been disclosed previously (see refs. 7, 8, 9, 10 and 11 and U.S. patent application Ser. No. 10/259,723 and patent application titled: “Nonlinear Modeling of Gene Networks From Time Series Gene Expression Data,” filed Nov. 18, 2003; Attorney Docket No: GENN 1008 US1 DBB, both applications incorporated herein fully by reference), as well as modeling gene expression data using an arbitrary system of differential equations (see Ref. 12). To reliably infer such an arbitrary system of differential equations, however, a long series of time-ordered gene expression data would be needed, which currently is often not yet available.

### SUMMARY

To overcome the disadvantages of the prior art, in certain aspects of this invention, we developed methods for inferring gene networks using a linear system of differential equations and information derived from gene expression data. This approach maintains the advantages of quantitativeness and causality inherent in differential equations, while being simple enough to be computationally tractable. We also developed new methods for testing hypotheses involving gene regulatory networks.

### BRIEF DESCRIPTION OF THE FIGURES

Aspects of this invention are described with reference to specific examples thereof. Other features of this invention can be understood by reference to the figures, in which:

FIG. 1 depicts a graph of gene expression of five clusters of genes from Bacillus subtilis with time.

FIG. 2 depicts a gene network, derived using methods of this invention, of the five clusters of genes depicted in FIG. 1.

### DETAILED DESCRIPTION

Modeling biological data using linear differential equations was considered theoretically by Chen (see Ref. 13). In this model, both the mRNA and the protein concentrations were described by a system of linear differential equations. Such a system can be described as

$$\frac{ⅆ}{ⅆ t} \underline{x} ( t ) = {Λ}_{\underline{\_}} \cdot \underline{x} ( t ) , \qquad ( 1 )$$

in which the vector x(t) contains the mRNA and protein concentrations as a function of time, and the matrix Λ is constant with units of [second]⁻¹. This equation can be considered as a generalization of the Boolean network model, in which the number of levels is infinite instead of binary.

In cDNA microarray experiments, usually only the gene expression levels are determined by measuring the corresponding mRNA concentrations, while the protein concentration is unknown. We therefore focus on a system of differential equations describing gene interactions only. A matrix element Λᵢⱼ then represents the effect of gene j on gene i, [Λᵢⱼ]⁻¹ being the reaction time.

To infer the coefficients in the system of differential equations from measured data, it was previously suggested (see Ref. 13) to discretize the system of differential equations, substitute the measured mRNA and protein concentrations, and solve the resulting linear system of equations to find the coefficients Λᵢⱼ in the system of linear differential equations. The system of equations is usually underdetermined. Using the additional requirement that the gene regulatory network should be sparse, Chen showed that the model can be constructed in O(m^{h+1}) time, where m is the number of genes and h is the number of nonzero coefficients allowed for each differential equation in the system (see Ref. 13).

Parameter h is chosen ad hoc, which has two unexpected consequences. As each row in the matrix Λ will have exactly h nonzero elements, every gene or protein in the network has h parent genes or proteins, and consequently no genes or proteins can exist at the top of a network. Secondly, every gene will inevitably be a member of a feedback loop. While feedback loops are likely to exist in gene regulatory networks, their existence should be determined from the measured data instead of created artificially.

Bayesian networks, on the other hand, do not allow the existence of loops. Bayesian networks rely on the joint probability distribution of the estimated network to be decomposable in a product of conditional probability distributions. This decomposition is possible only in the absence of loops. We further note that Bayesian networks tend to contain many parameters, and therefore need a large amount of data for a reliable estimation.

We therefore aimed to find methods that allow for the existence of loops in a network, but does not require their presence. Using Equation 1, we constructed a sparse matrix by limiting the number of nonzero coefficients that may appear in the system. Instead of choosing this number ad hoc, we estimated which coefficients in the interaction matrix are zero from the data by using Akaike's Information Criterion (AIC), allowing the number of gene regulatory pathways to be different for each gene.

Aspects of our method can be applied to find a network between individual genes, as well as a regulatory network between clusters of genes. As an example, one can infer a gene regulatory network between clusters of genes using time course data of Bacillus subtilis. Clusters can be created using the k-means clustering algorithm. The biological function of the clusters can be determined from the functional categories of the genes belonging to each cluster.

In some embodiments, we consider a regulatory network between m genes in terms of a linear system of differential equations (Equation 1), where the vector x(t) contains the expression ratios of the m genes at time t. This system of differential equations can be solved as

x(t)=exp[Λt]·x₀, (2)

in which x₀ contains the gene expression ratios at time zero. In this equation, the matrix exponential is defined in terms of a Taylor expansion as (see Ref. 14)

$$exp ( {A}_{\underline{\_}} ) ≡ {\sum }_{i = 0}^{∞} \frac{1}{i !} {A}^{i} . \qquad ( 3 )$$

As Equation 2 depends nonlinearly on Λ, it will be difficult to solve for Λ in terms of the measured data x(t). An approximate solution can be found by replacing the differential equation (Equation 1) by a difference equation:

$$\frac{Δ \underline{x}}{Δ t} = Λ \cdot \underline{x} , \qquad ( 4 )$$

or

x(t+Δt)−x(t)=Δt·Λ·x(t), (5)

which is of the form considered by Chen (see Ref. 13). To statistically determine the sparseness of matrix Λ, we explicitly add an error ε^{(t)}, which will invariably be present in the data:

x(t+Δt)−x(t)=Δt·Λ·x(t)+ε(t). (6)

By using this equation, we can effectively describe a gene regulatory network in terms of a multidimensional linear Markov model.

One can assume that the error has a normal distribution independent of time as shown below:

$$f ( \underline{ϵ} ( t ) ; {σ}^{2} ) = {( \frac{1}{\sqrt{2 π {σ}^{2}}} )}^{m} exp \{ - \frac{{\underline{ϵ} ( t )}^{T} \cdot \underline{ϵ} ( t )}{2 {σ}^{2}} \} , \qquad ( 7 )$$

with a standard deviation σ equal for all genes at all times. The log-likelihood function for a series of time-ordered measurements xᵢ at times tᵢ, i ∈{1, . . . , n} at n time points is then

$$L ( {Λ}_{\underline{\_}} , {σ}^{2} ) = - \frac{n m}{2} ln [ 2 π {σ}^{2} ] - \frac{1}{2 {σ}^{2}} {\sum }_{i = 1}^{n} {\hat{\underline{ϵ}}}_{i}^{T} \cdot {\hat{\underline{ϵ}}}_{i} , \qquad ( 8 )$$

in which

\hat{ε}ᵢ=xᵢ−xᵢ₋₁−(tᵢ−tᵢ₋₁)·Λ·xᵢ₋₁ (9)

is the measurement error at time tᵢ estimated from the measured data.

The maximum likelihood estimate of the variance σ² can be found by maximizing the log-likelihood function with respect to σ². This yields

$${\hat{σ}}^{2} = \frac{1}{n m} {\sum }_{i = 1}^{1} {\hat{\underline{ϵ}}}_{i}^{T} \cdot {\hat{\underline{ϵ}}}_{i} \cdot \qquad ( 10 )$$

Substituting this into the log-likelihood function (Equation 8) yields

$$L ( {Λ}_{\underline{\_}} , {σ}^{2} = {\hat{σ}}^{2} ) = - \frac{n m}{2} ln [ 2 π {\hat{σ}}^{2} ] - \frac{n m}{2} . \qquad ( 11 )$$

To find the maximum likelihood estimate \hat{Λ} of the matrix Λ we use Equation 9 to write the total squared error \hat{σ}² as

$${\hat{σ}}^{2} = \frac{1}{n m} {\sum }_{i = 1}^{n} [ ( {\underline{x}}_{i}^{T} - {\underline{x}}_{i - 1}^{T} ) \cdot ( {\underline{x}}_{i} - {\underline{x}}_{i - 1} ) + {( {t}_{i} - {t}_{i - 1} )}^{2} {\underline{x}}_{i - 1}^{T} \cdot {{Λ}_{\underline{\_}}}^{T} \cdot {Λ}_{\underline{\_}} \cdot {\underline{x}}_{i - 1} - 2 ( {\underline{x}}_{i}^{T} - ( {t}_{i} - {t}_{i - 1} ) {\underline{x}}_{i - 1}^{T} ) \cdot {Λ}_{\underline{\_}} \cdot {\underline{x}}_{i - 1} ] , \qquad ( 12 )$$

and take the derivative with respect to Λ. We find a linear equation in Λ:

\hat{Λ}B·A⁻¹, (13)

in which the matrices A and B are defined as

$$\begin{matrix}{A}_{\underline{\_}} ≡ {\sum }_{i = 1}^{n} [ {( {t}_{i} - {t}_{i - 1} )}^{2} \cdot {\underline{x}}_{i - 1} \cdot {\underline{x}}_{i - 1}^{T} ] ; & ( 14 ) \\ {B}_{\underline{\_}} ≡ {\sum }_{i = 1}^{n} [ ( {t}_{i} - {t}_{i - 1} ) \cdot ( {\underline{x}}_{i} - {\underline{x}}_{i - 1} ) \cdot {\underline{x}}_{i - 1}^{T} ] . & ( 15 )\end{matrix}$$

In the absence of errors, the estimated matrix \hat{Λ} is equal to the true matrix Λ. We know from biology that the gene regulatory network and therefore Λ is sparse. However, all of the elements in the estimated matrix \hat{Λ} may be nonzero due to the presence of noise, even if the corresponding elements in the true matrix Λ are zero.

In some embodiments, one can set a matrix element equal to zero if the resulting increase in the total squared error, as given by Equation 12, is small. Formally, we would use Akaike's Information Criterion (see Refs. 15 and 16)

$$AIC = 2 \cdot [ \begin{matrix}log \text{-} likelihood of the \\ estimated model\end{matrix} ] + 2 \cdot [ \begin{matrix}number of estimated \\ parameters\end{matrix} ] \qquad ( 16 )$$

to decide which matrix elements should be set equal to zero. The AIC can be used to avoid overfitting of a model to data by comparing the total error in the estimated model to the number of parameters that was used in the model. The model with the lowest AIC is considered to be optimal. The AIC is based on information theory and is widely used for statistical model identification, especially for time series model fitting (see Ref. 17).

We can then use a mask M to set matrix elements of \hat{Λ} equal to zero:

$${\hat{{Λ}_{\underline{\_}}}}^{′} = {M}_{\underline{\_}} ∘ {\hat{Λ}}_{\underline{\_}} , \qquad ( 17 )$$

where ∘ denotes the Hadamard (element-wise) product, (See Ref. 14) and the mask M is a matrix whose elements are either one or zero. The corresponding total squared error \hat{σ}² can be found by replacing \hat{Λ} by

$${{\hat{Λ}}_{\underline{\_}}}^{′}$$

in Equation 12. The total squared error, given the mask M, can be minimized by solving the set of equations

$$if {M}_{ij} = 1 {\text{:} [ {{\hat{Λ}}_{\underline{\_}}}^{′} • {A}_{\underline{\_}} ]}_{ij} = {B}_{ij} ; \\ if {M}_{ij} = 0 \text{:} {\hat{Λ}}_{ij} = 0 ; \qquad ( 18 )$$

yielding the maximum likelihood estimate

$${{\hat{Λ}}_{\underline{\_}}}^{′} .$$

In this equation, A and B are determined from Equations 14 and 15 using the measured gene expression levels xᵢ We then calculate the AIC corresponding to M by substituting the estimated log-likelihood function from Equation 11 into Equation 16:

AIC=nmln[2π\hat{σ}²]+nm+2·(1+[sum of the maskelements Mᵢⱼ]), (19)

the estimated parameters being \hat{σ}² and the elements of the matrix \hat{Λ} that we allow to be nonzero. From this equation, one can see that while the squared error decreases, the AIC may increase as the number of nonzero elements increases. A gene regulatory network may now be inferred from gene expression data by finding the mask M that yields the lowest value for the AIC.

For any but the most trivial cases, the number of possible masks M is extremely large, making an exhaustive search to find the optimal mask infeasible. Instead, one can use a greedy search method. Initially, one can choose a mask at random, with an equal probability of zero or one for each mask element. One can reduce the AIC by changing each of the mask elements Mᵢⱼ. This process can be continued until one finds a final mask for which no further reduction in the AIC can be achieved. This algorithm can be repeated starting from different (e.g., random) initial masks, and can be used to determine a final mask M that has the smallest corresponding AIC. If this optimal mask is found in several tens of trials, one can reasonably conclude that no better masks exist.

We have described and demonstrated methods to infer a gene regulatory network in the form of a linear system of differential equations from measured gene expression data. Due to the limited number of time points at which measurements are typically made, finding a gene regulatory network is usually an underdetermined problem. Since biologically the resulting gene regulatory network is expected to be sparse, we set some of the matrix entries equal to zero, and infer a network using only the nonzero entries. The number of nonzero entries, and thus the sparseness of the network, was determined from the data using Akaike's Information Criterion without using any ad hoc parameters.

Describing a gene network in terms of differential equations has at least three advantages. First, the set of differential equations describes causal relations between genes: a coefficient Λᵢⱼ of the coefficient matrix determines the effect of gene j on gene i. Second, it describes gene interactions in an explicitly numerical form. Third, because of the large amount of information present in a system of differential equations, other network forms can easily be derived from it. In addition, we can link the inferred network to other analysis or visualization tools, such as Genomic Object Net (see Ref. 22).

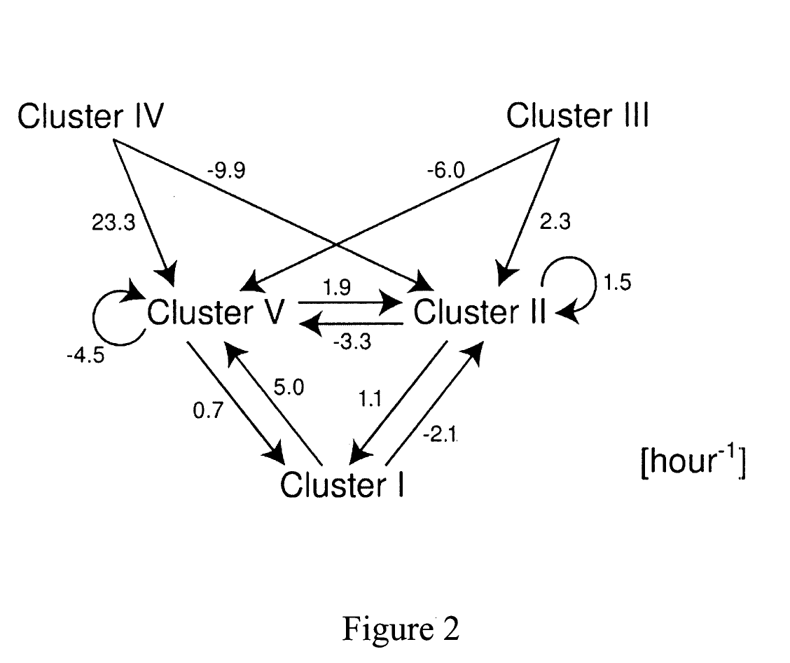

In previously described methods, either loops cannot be found (such as in Bayesian network models) or the methods artificially generate loops in the network. While the method described here allows loops to be present in the network, their existence is not required. Loops are found only if warranted by the data. For example, when inferring a regulatory network between gene clusters using time-course data of Bacillus subtilis in an MMGE medium, we found that some of the clusters were part of a loop, while others were not (see Examples below and FIG. 2).

If the number of genes m is equal to or larger than the number of experiments n, the matrix A in Equation 18 is singular. The problem is then underdetermined, and an interaction matrix \hat{Λ} can be found with zero total error \hat{σ}² and an AIC of \overline{}∞. This breakdown of our methods can be avoided by applying it to a sufficiently small number of genes or gene clusters, or by limiting the number of parents in the network.

Methods for Evaluating Statistical Significance of Network Relationships

In other embodiments of this invention, methods for determining statistical significance of analysis of network relationships are provided. Under the null hypothesis, one can hypothesize that a gene is not affected by the experimental manipulation. The measured log-ratios at different time points are then equivalent. We further can assume that the log-ratios have a normal distribution with zero mean. In some cases, a statistical test such as Student's t-test would be performed at every time point to determine which log-ratios are significantly different from zero. However, Student's t-test would be unreliable for data sets with only a few measurements. Therefore, in some embodiments including data sets having only two measurements at each time point, we devised a new statistical test, incorporating measurements at a plurality of time points. In particular, as shown in Example 2, we applied this method to data from all eight time points. It can be appreciated that the method can be used for other types of experiments, and will be described herein below.

Steps to carry out the method are described below.

Step 1: At each time point, calculate the average log-ratio as

$${\overline{x}}_{ji} = \frac{1}{2} {\sum }_{k = 1 , 2} {x}_{ji} [ k ] . \qquad ( 21 )$$

Under the null hypothesis, \overline{x}_{j•} (the average of two gene expression log-ratios at a time point) is a random variable with a normal distribution with zero mean and an estimated standard deviation, \hat{σ}_{j|H}_{₀}_{|./\sqrt{2}.}

Step 2: The standard deviation is then estimated from all measurements (e.g., 8×2=16 for the data set included as Example 1):

$${}^{\hat{σ}} j \vert {H}_{0} \vert . = \sqrt{\frac{1}{2 n} {\sum }_{i = 1}^{n} {\sum }_{k = 1 , 2} {( {x}_{ji} [ k ] )}^{2}} , \qquad ( 20 )$$

in which xᵢⱼ[k] denotes the data value of measurement k at time point i for gene j.

Step 3: The joint probability for \overline{x}_{j•} to be larger in absolute value than the measured values \overline{x}ⱼᵢ is then

$$\begin{matrix}P = {\prod }_{i = 1}^{n} {P}_{i} = {\prod }_{i = 1}^{n} p ( \vert {\overline{x}}_{j} • \vert > \vert {\overline{x}}_{ji} \vert ) \\ = {\prod }_{i = 1}^{n} [ 1 - erf ( \frac{\vert {\overline{x}}_{ji} \vert}{{}^{\hat{σ}} j \vert {H}_{0} \vert / \sqrt{2}} ) ] ,\end{matrix} \qquad ( 22 )$$

in which erf is the error function. For a single factor Pᵢ in this product, we would normally choose a significance level α, and reject the null hypothesis if Pᵢ<α

Step 4: Adopt a criterion that P<αⁿ for rejection of the null hypothesis. This allows one to determine whether the expression levels of a gene changed significantly during the experiment by making use of all the available data for that gene.

Step 5: Determine whether the expression levels of a gene change are significant.

The methods for determining network relationships between genes and the new statistical methods can be used in research, the biomedical sciences, including diagnostics, for developing new diagnoses and for selection of lead compounds in the pharmaceutical industry.

### EXAMPLES

The examples below are intended to illustrate embodiments of this invention, and are not intended to limit the scope. Other embodiments can be developed without departing from the scope of the invention, and methods of this invention and variants thereof can be used without undue experimentation to infer regulatory networks of different genes in B. subtilis and other organisms. All such embodiments are considered to be part of this invention.

### Example 1

### Gene Networks in Bacillus subtilis

Embodiments of this invention for finding a gene regulatory network using gene expression data were recently measured in an MMGE gene expression experiment of Bacillus subtilis (see Ref. 18). MMGE is a synthetic minimal medium containing glucose and glutamine as carbon and nitrogen sources. In this medium, the expression of genes required for biosynthesis of small molecules, such as amino acids, is induced. The expression levels of 4320 ORFs were measured at eight time points at one-hour intervals in this experiment, making two measurements at each time point.

Data Preparation and Analysis

To reduce the effect of measurement noise present in the data, the expression levels of each gene were compared to the measured background level. Genes with an average gene expression level lower than the average background level in either the red or the green channel were removed from the analysis.

Global normalization was then applied to the 3823 remaining genes, and the base-2 logarithms of the gene expression ratios were calculated. We applied a statistical test to the measured log-ratios to determine if they are significantly different from zero.

A flow chart for the method described above is reproduced in summary below.

Step 1: Calculate the average log-ratio of expression for each gene at each time point;

Step 2: Calculate the standard deviation from all measurements;

Step 3: Calculate the joint probability;

Step 4: Adopt a criterion for statistical significance; and

Step 5: Determine whether the expression levels of a gene change are significant.

In this Example, we chose a significance level α=0.00025 such that the expected number of false positives (0.00025×3823=1) was acceptable. By applying this criterion to the 3823 genes, we found that 684 genes were significantly affected.

### Example 2

### Clustering of Genes of B. subtilis

The 684 genes of B. subtilis were subsequently clustered into five groups using k-means clustering. The Euclidean distance was used to measure the distance between genes, while the centroid of a cluster was defined by the median over all genes in the cluster. The number of clusters was chosen such that a significant overlap was avoided. The k-means algorithm was repeated 1,000,000 times starting from different random initial clusterings. The optimal solution was found 81 times. The full clustering result is available at http://bonsai.ims.u-tokyo.ac.jp/˜mdehoon/publications/Subtilis/clusters.html.

In order to determine the biological function of the clusters that were created, we considered the functional category in the SubtiList database (see Refs. 19 and 20) for all genes in each cluster. Table 1 lists the main functional categories for the five clusters that were formed.

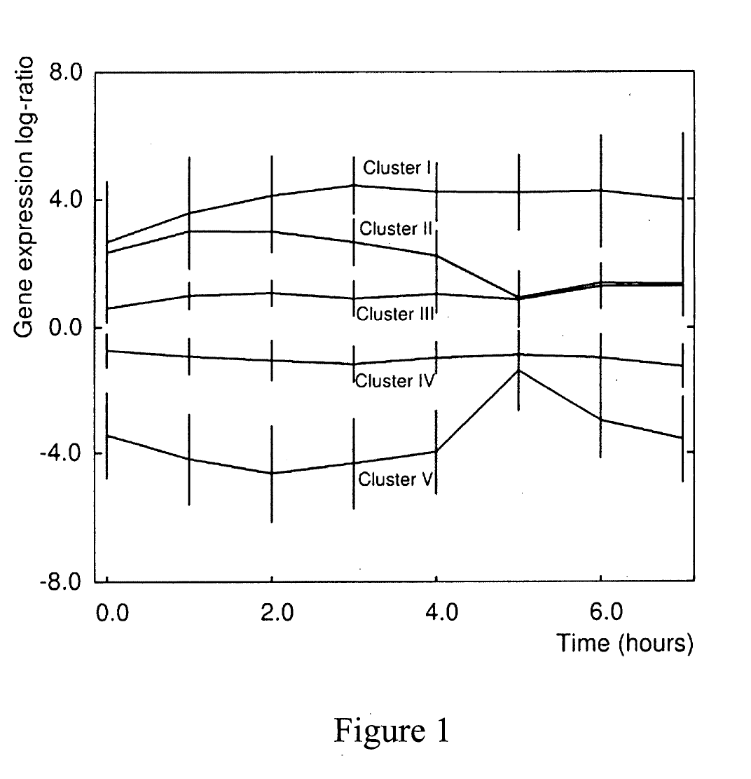

FIG. 1 shows the log-ratio of the gene expression as a function of time for each cluster. While the expression levels of clusters I, II, and V change considerably during the time course, clusters II and III have fairly constant expression levels. Cluster IV in particular can be considered as a catchall cluster, to which genes are assigned that do not fit well in the other clusters.

TABLE 1
Main functional categories for the five clusters created
using k -means clustering.

| Cluster | Number of genes | Main Functional Categories |
|---|---|---|
| I | 42 | 2.2: 11 genes; 1.1: 9 genes |
| II | 62 | 1.2: 15 genes; 2.2: 12 genes |
| III | 187 | 5.1: 30 genes; 6.0: 23 genes; 1.2: 22 genes |
| IV | 343 | 5.1: 40 genes; 5.2: 39 genes; 1.2: 33 genes |
| V | 50 | 1.2: 15 genes; 2.1.1: 15 genes |

### Functional Categories of Genes

Functional categories refer to the SubtiList database at Institut Pasteur.

| | |
|---|---|
| 1.1: | Cell wall. |
| 1.2: | Transport/binding proteins and lipoproteins. |
| 2.1.1: | Metabolism of carbohydrates and related molecules |
| | Specific pathways. |
| 2.2: | Metabolism of amino acids and related molecules. |
| 5.1: | Similar to unknown proteins from Bacillus subtilis. |
| 5.2: | Similar to unknown proteins from other organisms. |
| 6.0: | No similarity. |

FIG. 1 shows the log-ratio of the gene expression as a function of time for each cluster, as determined from the measured gene expression data.

Subsection Network Construction

From the measured log-ratios of those twelve genes, we constructed the matrices A and B and calculated the matrix \hat{Λ}. The process of calculating a mask M, starting from a random initial mask, was repeated 1000 times. The optimal solution was found 55 times. It is therefore unlikely that there are other masks with a lower AIC. Note that total number of possible mask is 2²⁵=33,554,432.

The network that was found is shown in FIG. 2. The number of parents of a cluster in the network varies between zero and five. Clusters III and IV appear as the top of the network, while clusters I, II and V are connected in a loop. Note that this network can neither be generated by the previously proposed method (see Ref. 13), nor by a Bayesian network model.

The two strongest interactions in the network are the positive and negative effect of cluster IV on cluster V and cluster II respectively. The opposite behaviors of the gene expression levels of clusters II and V are most likely caused by cluster IV, instead of a direct interaction between clusters II and V.

FIG. 2 shows the network between the five gene clusters, as determined from the MMGE time-course data and methods of this invention. The values show how strongly one gene cluster affects another gene cluster, as given by the corresponding elements in the interaction matrix

$${{\hat{Λ}}_{\underline{\_}}}^{′} .$$

In effect, this matrix represents how rapidly gene expression levels respond to each other. As an example, a change in the gene expression level of Cluster I would cause the expression level of Cluster V to change considerably within 1/(5.0 hour⁻¹)=12 minutes, if the expression levels of Clusters II, III, and IV are unchanged.

### References

1. P. T. Spellman, G. Sherlock, M. Q. Zhang, V. R. Iyer, K. Anders, M. B. Eisen, P. O. Brown, D. Botstein, and B. Futcher, “Comprehensive identification of cell cycle-regulated genes of the yeast Saccharomyces cerevisiae by microarray hybridization” Mol. Biol. Cell 9 (1998) 3273-3297.

2. J. L. DeRisi, V. R. Iyer, and P. O. Brown, “Exploring the metabolic and genetic control of gene expression on a genomic scale” Science 278 (1997) 680-686.

3. Y. Hihara, A. Kamei, M. Kanehisa, A. Kaplan, and M. Ikeuchi, “DNA microarray analysis of cyanobacterial gene expression during acclimation to high light” The Plant Cell 13 (2001) 793-806.

4. M. J. L. de Hoon, S. Imoto, and S. Miyano, “Statistical analysis of a small set of time-ordered gene expression data using linear splines” Bioinformatics, in press.

5. M. B. Eisen, P. T. Spellman, P. O. Brown, and D. Botstein, “Cluster analysis and display of genome-wide expression patterns” Proc. Natl. Acad. Sci. USA 95 (1998) 14863-14868.

6. P. Tamayo, D. Slonim, J. Mesirov, Q. Zhu, S. Kitareewan, E. Dmitrovsky, E. S. Lander, and T. R. Golub, “Interpreting patterns of gene expression with self-organizing maps: Methods and application to hematopoietic differentiation” Proc. Natl. Acad. Sci. USA 96 (1999) 2907-02912.

7. S. Liang, S. Fuhrman, and R. Somogyi, “REVEAL, a general reverse engineering algorithm for inference of genetic network architectures” Proc. Pac. Symp. on Biocomputing 3 (1998) 18-29.

8. T. Akutsu, S. Miyano, and S. Kuhara, “Inferring qualitative relations in genetic networks and metabolic pathways” Bioinformatics 16 (2000) 727-734.

9. N. Friedman, M. Linial, I. Nachman, and D. Pe'er, “Using Bayesian networks to analyze expression data” J. Comp. Biol. 7 (2000) 601-620.

10. S. Imoto, T. Goto, and S. Miyano, “Estimation of genetic networks and functional structures between genes by using Bayesian networks and nonparametric regression” Proc. Pac. Symp. on Biocomputing 7 (2002) 175-186.

11. S. Imoto, S. -Y. Kim, T. Goto, S. Aburatani, K. Tashiro, S. Kuhara, and S. Miyano, “Bayesian network and nonparametric heteroscedastic regression for nonlinear modeling of genetic network” Proc. IEEE Computer Society Bioinformatics Conference (2002) 219-227.

12. E. Sakamoto and H. Iba, “Evolutionary inference of a biological network as differential equations by genetic programming” Genome Informatics 12 (2001) 276-277.

13. T. Chen, H. L. He, and G. M. Church, “Modeling gene expression with differential equations” Proc. Pac. Symp. on Biocomputing 4 (1999) 29-40.

14. R. A. Horn and C. R. Johnson, Matrix Analysis. Cambridge University Press, Cambridge, UK (1999).

15. H. Akaike, “Information theory and an extension of the maximum likelihood principle” Research Memorandum No. 46, Institute of Statistical Mathematics, Tokyo (1971). In B. N. Petrov and F. Csaki (editors), 2nd Int. Symp. on Inf. Theory. Akadémiai Kiadó, Budapest (1973) 267-281.

16. H. Akaike, “A new look at the statistical model identification” IEEE Trans. Automat. Contr. AC-19 (1974) 716-723.

17. M. B. Priestley, Spectral Analysis and Time Series. Academic Press, London (1994).

18. Microbial Advanced Database Organization (Micado). http://www-mig.versailles.inra.fr/bdsi/Micado/.

19. I. Moszer, P. Glaser, and A. Danchin, “SubtiList: a relational database for the Bacillus subtilis genome” Microbiology 141 (1995) 261-268.

20. I. Moszer, “The complete genome of Bacillus subtilis: From sequence annotation to data management and analysis” FEBS Letters 430 (1998) 28-36

21. T. W. Anderson and J. D. Finn, The New Statistical Analysis of Data. Springer Verlag, New York (1996).

22. H. Matsuno, A. Doi, Y. Hirata, and S. Miyano, “XML documentation of biopathways and their simulation in Genomic Object Net” Genome Informatics 12 (2001) 54-62. Genomic Object Net is available at http://www.GenomicObject.net.

We claim:

1. A method for inferring a network relationship between genes, comprising:
(a) providing a quantitative time course data library for a set of genes of an organism, said library including expression results based on time course of expression of each gene in said set of genes, quantifying an average effect and measure of variability of each time point on each other of said genes;
(b) creating a sparse matrix from said library, said matrix having zero coefficients removed therefrom;
(c) generating a set of linear differential equations from said matrix; and
(d) solving said set of equations to produce said network relationship.

2. The method of claim 1, wherein said zero coefficients are identified using Akaike's Information Criterion (AIC).

3. The method of claim 1, wherein said differential equation is
$$\frac{ⅆ}{ⅆ t} \underline{x} ( t ) = {Λ}_{\underline{\_}} • \underline{x} ( t ) ,$$
in which the vector ^{x(t)} contains the amount of expressed cDNA as a function of time, and the matrix ^{Λ} is a constant with units second⁻¹.

4. The method of claim 1, wherein said matrix contains elements Λᵢⱼ, wherein Λᵢⱼ represents the effect of gene j on gene i, and wherein [Λᵢⱼ]⁻¹ represents the reaction time for said effect of gene j on gene i.

5. The method of claim 1, wherein said differential equation solved is
x(t)=exp[Λ^{t}]·x₀,)

6. The method of claim 1, wherein said exponent Λt (exp(Λ)) is solved using the formula:
$$exp ( {A}_{\underline{\_}} ) ≡ {\sum }_{i = 0}^{∞} \frac{1}{i !} {{A}_{\underline{\_}}}^{i} .$$

7. The method of claim 1, wherein said differential equation is estimated by solving the difference equation:
$$\frac{Δ \underline{x}}{Δ t} = \underline{\underline{Λ}} \cdot \underline{x} ,$$

8. The method of claim 1, wherein said sparse matrix further comprises an error estimated using, the formula:
x(t+Δt)−x(t)=Δt·Λ·x(t)+ε(t).

9. The method of claim 8, wherein said error has a normal distribution independent of time according to the formula:
$$f ( \underline{ϵ} ( t ) ; {σ}^{2} ) = {( \frac{1}{\sqrt{2 {πσ}^{2}}} )}^{m} exp \{ - \frac{{\underline{ϵ} ( t )}^{T} \cdot \underline{ϵ} ( t )}{2 {σ}^{2}} \} ,$$
wherein standard deviation a is equal for each of said genes at all times.

10. The method of claim 1, wherein the maximum likelihood estimate of the variance σ² is determined by maximizing the log-likelihood function with respect to σ² using the formula:
$${\hat{σ}}^{2} = \frac{1}{nm} {\sum }_{i = 1}^{1} {\hat{\underline{ ϵ}}}_{i}^{T} \cdot {\underline{\hat{ϵ}}}_{i} \cdot$$

11. The method of claim 10, wherein said variance σ² is determined using the formula:
$${\hat{σ}}^{2} = \frac{1}{nm} {\sum }_{i = 1}^{n} [ ( {\underline{x}}_{i}^{T} - {\underline{x}}_{i - 1}^{T} ) \cdot ( {\underline{x}}_{i} - {\underline{x}}_{i - 1} ) + {( {t}_{i} - {t}_{i - 1} )}^{2} {\underline{x}}_{i - 1}^{T} \cdot {\underline{\underline{Λ}}}^{T} \cdot \underline{\underline{Λ}} \cdot {\underline{x}}_{i - 1} - {2}_{′} ( {\underline{x}}_{i}^{T} - ( {t}_{i} - {t}_{i - 1} ) {\underline{x}}_{i - 1}^{T} ) \cdot \underline{\underline{Λ}} \cdot {\underline{x}}_{i - 1} ] ,$$

12. The method of claim 2, wherein said AIC is minimized using the formula:
$$AIC = 2 \cdot [ \begin{matrix}log - likelihood of the \\ estmated model\end{matrix} ] + 2 \cdot [ \begin{matrix}number of estimated \\ parameters\end{matrix} ] \qquad ( 16 )$$

13. The method of claim 1, wherein mask ^{M} is used to set matrix elements of ^{\hat{Λ}} equal to zero using the formula:
$${\hat{\underline{\underline{Λ}}}}^{′} = \underline{\underline{M} } ° \underline{\underline{\hat{Λ}}} ,$$
where ∘ denotes an element-wise product, and mask ^{M} is a matrix whose elements are either one or zero.

14. The method of claim 13, wherein matrix elements are set to zero by applying a mask M produced by minimizing the formula:
$$\begin{matrix}if {M}_{ij} = 1 : {[ {\underline{\hat{\underline{Λ}}}}^{′} \cdot \underline{\underline{A}} ]}_{ij} = {B}_{ij} ; \\ if {M}_{ij} = 0 : {{\hat{Λ}}^{′}}_{ij} = 0 ;\end{matrix}$$
thereby yielding the maximum likelihood estimate
$${\underline{\underline{\hat{Λ}}}}^{′} .$$

15. The method of claim 2, wherein said AIC is minimized according to the formula:
AIC=nm 1n [2π\hat{σ}²]+nm+2·(1+[sum of the maskelements Mᵢⱼ]),

16. The method of claim 13, wherein said mask M is selected to minimize AIC calculated using the formula:
AIC=nm ln [2π\hat{σ}²]+nm+2·(1+[sum of the maskelements Mᵢⱼ]),

17. A medium containing one or more results of network relationships between genes calculated using a method of claim 1 stored thereon.

18. A method for determining the statistical significance of network relationships, comprising:
(a) calculating the average log-ratio of expression for each gene at each time point;
(b) calculating the standard deviation from all measurements;
(c) calculate the joint probability; and
(d) adopting a criterion for statistical significance.

19. The method of claim 18, wherein said step (a) is determined using the formula:
$${\overline{x}}_{ji} = \frac{1}{2} {\sum }_{k = 1 , 2} {x}_{ji} [ k ] .$$

20. The method of claim 18, wherein step (b) is determined using the formula:
$${\hat{σ}}_{j \vert {H}_{0} \vert} = \sqrt{\frac{1}{2 n} {\sum }_{i = 1}^{n} {\sum }_{k = 1 , 2}^{} {( {x}_{ji} [ k ] )}^{2} ,}$$
in which ^{x}^{ⱼᵢ}^{[k]} is the data value of measurement k at time point i for gene j.

21. The method of claim 18, wherein the joint probability for \overline{x}_{j•} to be larger in absolute value than the measured values \overline{x}ⱼᵢ is calculated using the formula:
$$P = {\prod }_{i = 1}^{n} {P}_{i} = {\prod }_{i = 1}^{n} p ( \vert {\overline{x}}_{j \cdot } \vert > \vert {\overline{x}}_{ji} \vert ) = {\prod }_{i = 1}^{n} [ 1 - erf ( \frac{\vert {\overline{x}}_{ji} \vert}{{\hat{σ}}_{j \vert {H}_{0} \vert / \sqrt{2}}} ) ] ,$$
wherein erf is an error function.

22. The method of claim 18, wherein a significance level α is selected.

23. The method of claim 18, wherein the null hypothesis is rejected if Pi<α.

24. The method of claim 18, wherein the null hypothesis is rejected if P<αⁿ, wherein n is the number of time points at which gene expression is evaluated.

25. A method for determining the statistical significance of network relationships, comprising:
(a) calculating the average log-ratio of measurements of expression for each gene at each time point using the formula:
$${\overline{x}}_{ji} = \frac{1}{2} {\sum }_{k = 1 , 2} {x}_{ji} [ k ] .$$
(b) calculating the standard deviation of said measurements using the formula:
$${\hat{σ}}_{j \vert {H}_{0} \vert = \sqrt{\frac{1}{2 n} {\sum }_{i - 1}^{n} {\sum }_{k = 1 , 2} {( {x}_{ji} [ k ] )}^{2} ,}}$$
in which ^{x}^{ⱼᵢ}^{[k]} is the data value of measurement k at time point i for gene j.
(c) calculating a joint probability for \overline{x}_{j•} to be larger in absolute value than measured values \overline{x}ⱼᵢ calculated using the formula:
$$P = {\prod }_{i = 1}^{n} {P}_{i} = {\prod }_{i = 1}^{n} p ( \vert {\overline{x}}_{j \cdot } \vert > \vert {\overline{x}}_{ji} \vert ) = {\prod }_{i = 1}^{n} [ 1 - erf ( \frac{\vert {\overline{x}}_{ji} \vert}{{\hat{σ}}_{j \vert {H}_{0} \vert / \sqrt{2}}} ) ] ,$$
wherein erf is an error function; and
(d) applying a criterion for statistical significance to determine whether a null hypothesis is rejected.

26. The method of claim 25, wherein the null hypothesis is rejected if P<αⁿ, wherein n is the number of time points at which gene expression is evaluated.
